(19) United States
(12) Patent Application Publication
(10) Pub. No.: US 2016/0157905 A1
(43) Pub. Date: Jun. 9, 2016

(54) FLEXIBLE LOCKED PLATE FIXATION

(71) Applicant: Stryker European Holdings I, LLC, Kalamazoo, MI (US)

(72) Inventors: Luis A. Arellano, Fair Lawn, NJ (US); Eric Chang, East Brunswick, NJ (US)

(21) Appl. No.: 15/047,175

(22) Filed: Feb. 18, 2016

Related U.S. Application Data
(62) Division of application No. 13/655,578, filed on Oct. 19, 2012.
(60) Provisional application No. 61/549,439, filed on Oct. 20, 2011.

(51) Int. Cl. A61B 17/80

(52) U.S. Cl. CPC A61B 17/8085; A61B 17/8047

(57) ABSTRACT

A bone fixation device is disclosed that permits some movement of different portions of a fractured bone when the device is affixed thereto. Various embodiments are disclosed, including bone plates that have fixed holes and floating holes therethrough for receiving bone screws, and bone plates including oblong holes allowing for screw movement.

### CROSS-REFERENCE TO RELATED APPLICATIONS

The present application claims the benefit of the filing date of U.S. Provisional Patent Application No. 61/549,439 filed on Oct. 20, 2011, the disclosure of which is hereby incorporated herein by reference.

### BACKGROUND OF THE INVENTION

The present invention relates to devices used in the fixation of fractures in bones. More specifically, the present invention relates to a flexible bone plating system that rigidly fixes adjacent ends of a fractured bone while still allowing axial dynamization of the bone to promote healing of the fracture.

Typical fixation of a fracture of a long bone with a bone plate requires making an incision in the tissue, reducing the fracture, placing a bone plate on the fractured bone, and securing the bone plate to the bone with fixation elements such as screws. The bone plate immobilizes the fracture and keeps the bone in a correct position so as to allow the fracture to heal. In certain cases, the bone plate may facilitate the reduction of the fracture.

Typically, bone plates have a bone contacting surface and an upper surface facing away from the bone with a plurality of holes or apertures extending between the two surfaces. These holes or apertures may be either threaded (for use with locking screws) or non-threaded (for use with regular screws) and may be circular or oblong in shape. In traditional fracture fixation, the plates are usually fixed to the bone parts by means of threaded screws, which are driven into the bone tissue after so-called pre-drilled or pilot-drilled holes have been generated in the bone tissue. These pre-drilled holes allow for a reliable screwing procedure whereby the risk of further destroying the bone with the screw is significantly reduced. These plates generally are rigid and resistant to bending or torsioning in order to stabilize the fractured bone. However, absolute rigidity is not always desirable and it can be advantageous when an implantable bone plate is capable of a degree of freedom along the main axis of the bone plate.

### BRIEF SUMMARY OF THE INVENTION

Various aspects of the present invention are achieved by bone fixation devices, screws, or drilling configurations that result in the capability of axial dynamization of the bone along the main axis of the bone while maintaining rigidity in other directions and restricting bending or torsioning.

In one embodiment, a bone fixation device, such as a bone plate, for affixation to a bone has a first end, a second end remote from the first end, a bone contacting surface and an upper surface facing away from the bone contacting surface. The bone fixation device also has first and second holes extending between the bone contacting surface and the upper surface, with the first hole positioned on the first end, the second hole positioned on the second end, and each hole being capable of receiving a fastener. The bone fixation device also includes a nut with a top surface, a bottom surface, and a hole extending between the top surface and bottom surface.

The first hole may be oblong with a major axis length and a minor axis length. The nut can have a width greater than the major axis length of the first hole. Alternatively, the nut can have a top portion and a bottom portion. The width of the top portion is smaller than the major axis length of the first hole and the width of the bottom portion is greater than the major axis length of the first hole.

In another embodiment, a bone fixation device, such as a bone plate, for affixation to a bone includes a first end, a second end remote from the first end, a bone contacting surface, and an upper surface facing away from the bone contacting surface. The bone fixation device also includes first and second holes extending between the bone contacting surface and the upper surface, the first hole being positioned on the first end the second hole being positioned on the second end. Each hole is capable of receiving a fastener. The bone fixation device can also include a cap with a top surface, a bottom surface, a first side wall, and a second side wall. The bottom surface of the cap defines a recess therein. The first and second side walls of the cap can each include a flange extending generally parallel to the top surface toward the opposite side wall.

In another embodiment of the invention, a method of affixing a bone fixation device to a bone with a fracture includes the step of providing the bone fixation device. The bone fixation device includes a first end, a second end remote from the first end, a bone contacting surface and an upper surface facing away from the bone contacting surface. The bone fixation device also includes first and second holes extending between the bone contacting surface and the upper surface, the first hole being positioned on the first end the second hole being positioned on the second end. Each hole is capable of receiving a fastener. The bone fixation device also includes a nut with a top surface, a bottom surface, and a hole extending between the top surface and bottom surface. This embodiment of the invention also includes the steps of placing the bone fixation device on the bone, inserting a first fastener through the first hole, through the hole in the nut, and into the bone on a first side of the fracture, and also inserting a second fastener through the second hole, and into the bone on a second side of the fracture.

In yet another embodiment of the invention, a method of affixing a bone fixation device to a bone with a fracture includes the step of providing the bone fixation device, which includes a first end, a second end remote from the first end, a bone contacting surface and an upper surface facing away from the bone contacting surface. The bone fixation device also includes first and second holes extending between the bone contacting surface and the upper surface, the first hole being positioned on the first end and the second hole being positioned on the second end, each hole being capable of receiving a fastener. The bone fixation device also includes a cap with a top surface, a bottom surface, first and second side walls, with the bottom surface of the cap defining a recess therein. This embodiment of the invention also includes the steps of placing the bone fixation device on the bone, inserting first and second fasteners through the first and second holes and into the bone on first and second sides of the fracture, respectively. This embodiment of the invention also includes the step of connecting the cap to the bone fixation device.

The step of connecting the cap to the bone fixation device can also include placing the cap over the first hole such that at least a portion of the first fastener resides within the recess of the bottom surface of the cap. The step of connecting the cap to the bone fixation device can even further include engaging a flange on the first side wall of the cap with the bone contacting surface of the bone fixation device and engaging a flange on the second side wall of the cap with the bone contacting surface of the bone fixation device.

Different embodiments of a bone fixation device include apertures into which screws are inserted to fix the bone fixation device to the bone on one side of a fracture site, and further include additional apertures that can receive screws on the opposing side of the fracture site. The additional apertures can have a means by which the screw and aperture can slightly move in a direction along the main axis of the bone while the screw remains fixed in the bone. This preferably allows for micromotion of the bone fragments on each side of the fracture to promote healing.

Different embodiments of the means by which the screw and aperture can move are within the scope of the invention. These include, for example, a floating aperture element in the bone fixation device created by cutting out material from the bone fixation device, leaving the floating aperture connected by flexure joints or another type of spring mechanism. Other means contemplated herein include an aperture made within a sliding cart in the bone fixation device which allows the screw and the sliding cart to slide within a predefined area of the bone fixation device, while the screw engaged with the aperture remains fixed relative to the segment of bone into which it is inserted.

Other embodiments of a bone fixation device to achieve the desired goals include a bone fixation device that includes flexible arm extensions, forming a cut-out section in the center, that connect opposing ends of the bone fixation device and allow the bone fixation device to flex in the direction of the main axis of the bone while maintaining rigidity in other directions.

In another embodiment, the bone fixation device includes at least one oblong hole into which a bone screw can be inserted. The oblong shape of the hole allows movement of the screw along the major axis of the oblong hole, while tilting or movement in other directions is restricted by the minor axis of the oblong hole as well as other means. Examples of the other means contemplated herein include a nut, through which the screw is inserted, which at least partially contacts the underside of the bone fixation device to provide rigidity in the desired directions. Further means include a cap and screw assembly in which the screw can move with the bone in the direction of the main axis of the bone while still being restricted from movement in other directions by the cap.

Still further embodiments of the invention are provided to achieve axial dynamization of a bone. These include screw configurations in which a top portion of the screw is narrower than a bottom portion of the screw, due to either the core diameter or the thread diameter changing along the axis of the screw. This geometry allows for reduced rigidity on the near cortex side of the bone after the screw is inserted through an aperture in a bone fixation device and further through the bone. The reduced rigidity allows the screw and bone to move along the main axis of the bone while restricting movements in other directions. Similarly, an embodiment of the invention achieves a similar result using a screw with a constant core and thread diameter, but instead creating a relatively large drill hole in the near cortex and relatively small drill hole in the far cortex of the bone.

### BRIEF DESCRIPTION OF THE DRAWINGS

FIG. 1A illustrates a top view of one embodiment of a bone fixation device shown here as a bone plate.

FIG. 1B illustrates an enlarged view of a floating aperture element of the embodiment of the bone fixation device in FIG. 1A.

FIG. 2A illustrates a top view of an alternate embodiment of a bone fixation device in the form of a bone plate.

FIG. 2B illustrates a cross section of a cart element of the embodiment of the bone fixation device in FIG. 2A along axis A-A.

FIG. 2C illustrates a perspective view of an alternate embodiment of a bone fixation device.

FIG. 3 illustrates yet another embodiment of a bone fixation device, shown as a bone plate with flexible arm extensions.

FIG. 4A illustrates a top view of another embodiment of a bone fixation device, shown as a bone plate with an oblong screw hole.

FIG. 4B illustrates a cross-sectional side view of a screw engaged with a nut through the oblong screw hole of the bone fixation device shown in FIG. 5A.

FIG. 4C illustrates a cross-sectional side perspective view of a screw engaged in the oblong screw hole of the bone fixation device shown in FIG. 5A along with a screw cap.

FIG. 4D illustrates a bottom perspective view of the screw cap shown in FIG. 5C.

FIG. 5 illustrates a cross sectional view, along the main axis of a bone, of a bone after different diameter holes are drilled through the bone.

### DETAILED DESCRIPTION

As used herein, when referring to bones or other parts of the body, the term “proximal” means closer to the heart and the term “distal” means more distant from the heart. The term “inferior” means toward the feet and the term “superior” means towards the head. The term “anterior” means towards the front part of the body or the face and the term “posterior” means towards the back of the body. The term “medial” means toward the midline of the body and the term “lateral” means away from the midline of the body.

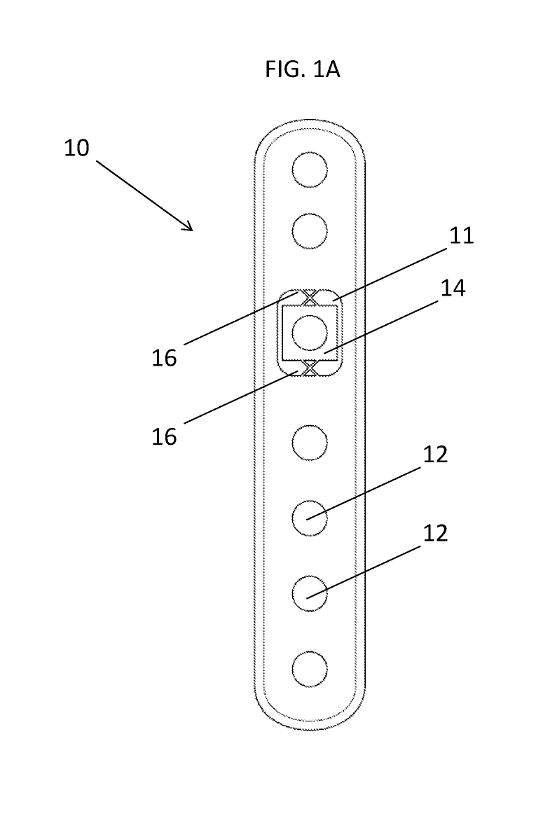

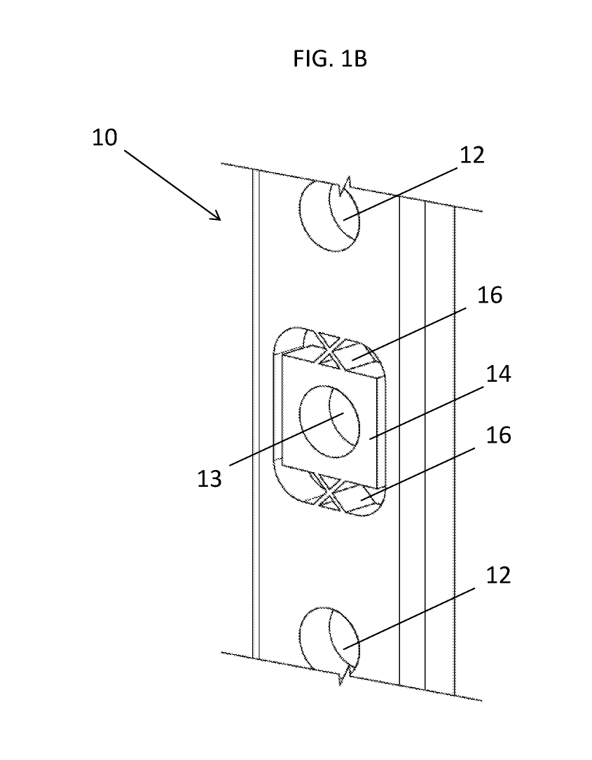

Referring to FIG. 1A there is shown a top view of one embodiment of bone fixation device 10. Bone fixation device 10 includes a bone contacting surface (not shown) and a surface facing away from the bone (shown in FIG. 1) with a plurality of holes or apertures 12 extending between the two surfaces. Holes or apertures 12 may be either threaded (for use with locking screws) or non-threaded (for use with non-locking or compression screws) and may be any suitable shape, such as circular or oblong. Bone fixation device 10 is implanted onto a bone across a fracture site in the bone such that at least one hole or aperture 12 is positioned on each opposing side of the fracture. Bone fixation device 10 also includes one or more floating aperture elements 14, within a slot 11, connected to bone fixation device 10 by flexure joints 16, described in more detail below.

Referring to FIG. 1B there is shown a close up view of floating aperture element 14 within a slot 11 of bone fixation device 10. Each of the one or more slots 11 are created by removing material from the bone fixation device 10 around a hole or aperture 13. The remaining material forms the one or more floating aperture elements 14. The material is removed such that the floating aperture element 14 remains connected to the bone fixation device 10 by flexure joints 16 to allow the floating aperture element 14 to move only in the desired direction (e.g., along the main or longitudinal axis of the bone fixation device). As shown, the floating aperture element 14 is generally a rectangular shape that only contacts the bone fixation device 10 along the main axis of the device 10. The flexure joints 16 are generally “x” shaped and integral with the bone fixation device 10 such that the floating aperture element 14 can move along the longitudinal axis of the bone fixation device 10, even while the rest of the bone fixation device 10 is otherwise stationary. Thus, the flexure joints 16 allow the floating aperture element 14 to be stiff in some directions and flexible in others. By leveraging this asymmetric stiffness, a change in the distance between holes 12 and 13 is possible with minimal effect on the angle between the bone fixation device 10 and the bone to which it is attached. Additionally, there is minimal allowance for horizontal translation of a screw in aperture 13 as well as minimal allowance for translation of the screw in a direction normal to the bone fixation device 10.

Although the embodiment depicted in FIGS. 1A and 1B is discussed above, one skilled in the art would recognize that design variations in the fixation device are possible. For instance, floating aperture element 14 could be other shapes such as square or circular, and the flexure joints 16 could be any shape, such as a spring shape that allows translation in the axial direction while maintaining stiffness in other directions. Likewise, the fixation device can exhibit many different shapes for use in different situations, and can employ any number of floating aperture elements 14 and slots 11. Still further, floating aperture elements 14 and flexure joints 16 can be configured to allow translation or movement of the screw in a number of different directions, as well as to allow more or less movement. Although discussed as being created through the removal of material from device 10, floating apertures elements 14 could be separately formed and attached to device 10 through welding or another suitable process.

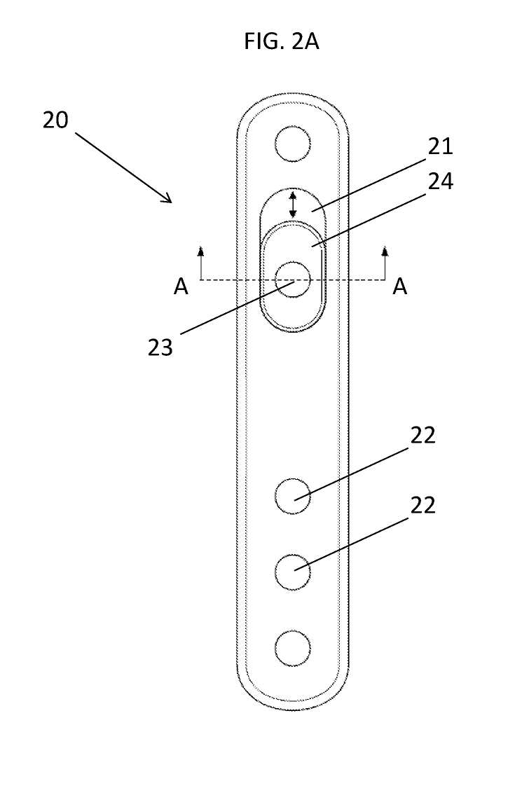

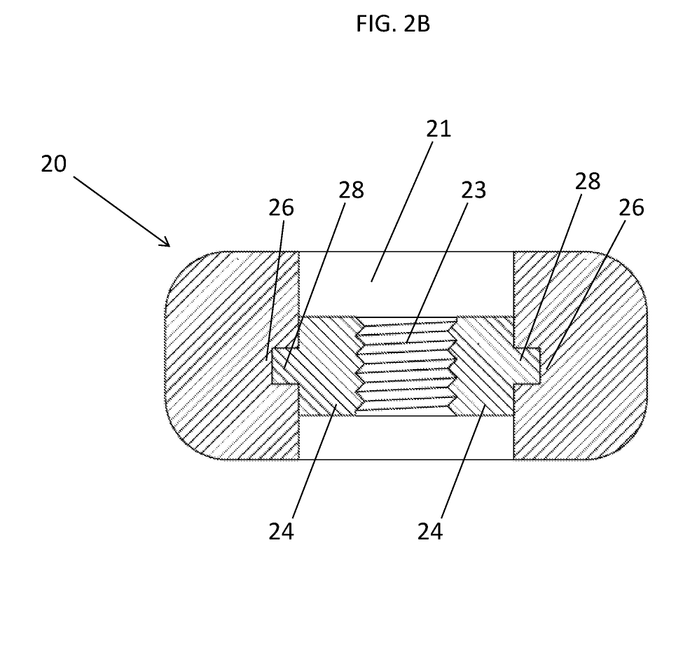

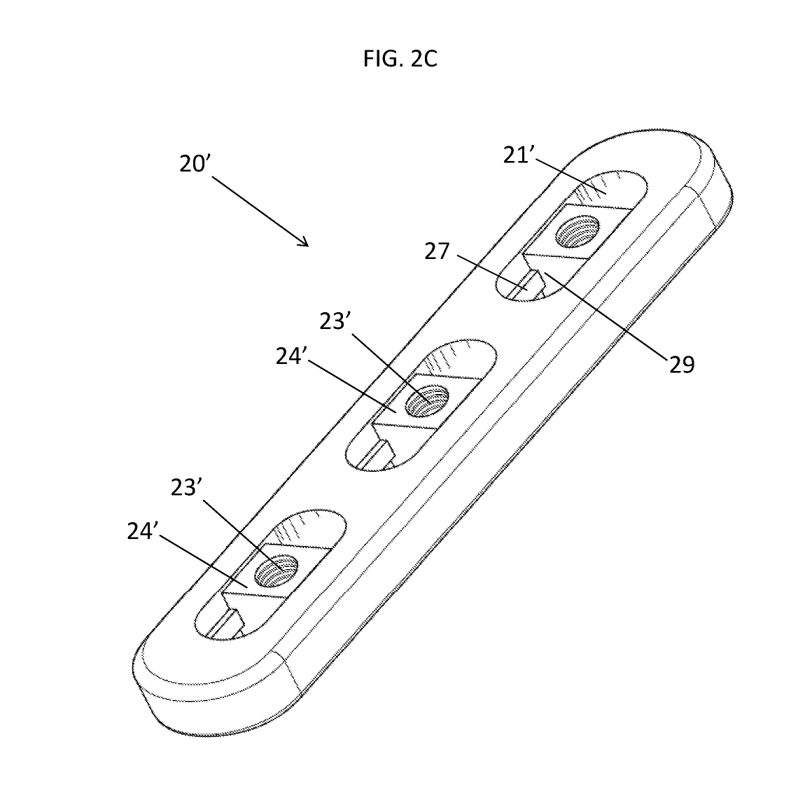

Referring to FIG. 2A there is shown a top view of an alternate embodiment of a bone fixation device 20. This embodiment is similar to that illustrated in FIG. 1A in many respects, with the main difference being the inclusion of a cart element 24. Bone fixation device 20 includes a bone contacting surface and an upper surface facing away from the bone with a plurality of holes or apertures 22 extending between the two surfaces. Holes or apertures 22 may be either threaded (for use with locking screws) or non-threaded (for use with non-locking or compression screws) and may be any suitable shape, such as circular or oblong. Bone fixation device 20 is implanted onto a bone across a fracture site in the bone such that at least one hole or aperture 22 is positioned on each opposing side of the fracture. Bone fixation device 20 also includes one or more slots 21. Each of the one or more slots 21 may include a cart element 24 guided by railing slots 26 of the bone fixation device 20, described in more detail below. Each cart element 24 may be provided as a separate insert. This allows the surgeon to insert the cart element 24 into the respective slot 21 prior to or during surgery based on the surgeon's assessment of the most appropriate course of action at the time.

Referring to FIG. 2B, there is shown a cross section of the embodiment of bone fixation device 20 in FIG. 2A along axis A-A. As shown, each cart element 24 includes a hole or aperture 23 therein. Each cart element 24 also includes protrusions 28 that fit into railing slots 26 of the bone fixation device 20. The fit between the railing slots 26 and protrusions 28 is such that the cart element 24 may slide along the length of the railing slots 26. When a screw is fixed into a hole or aperture 22 on one side of a bone fracture and another screw is fixed in hole or aperture 23 of cart element 24, a small degree of motion in the main or longitudinal axis of the bone fixation device 20 is possible due to the sliding capability of the cart element 24 without the introduction of shear or bending forces, which may be undesirable or detrimental to uniform bone growth. Although the cart element 24 is illustrated as having a smaller thickness than the bone fixation device 20, the cart element may be thinner or thicker than illustrated. For example, the cart element 24 may alternately be approximately the same thickness as the bone fixation device 20.

Referring to FIG. 2C, there is shown a perspective view of yet another embodiment of bone fixation device 20′. This embodiment of the bone fixation device 20′ has multiple cart elements 24′, each with a threaded hole or aperture 23′. As with other embodiments of the current invention, the holes or apertures may be threaded or unthreaded, and there may be one or more cart elements 24′ included with the bone fixation device. In this embodiment, as opposed to that shown in FIGS. 2A-B, the cart element 24′ includes one or more cart slots 29 that each mate with a protruding railing 27 formed in slots 21′. Similar to the embodiment shown in FIGS. 2A-B, the cart element 24′ can freely slide inside bone fixation device slot 21′, the cart element 24′ being guided by protruding railing 27 allowing motion only along the main or longitudinal axis of the bone fixation device 20. As in the above embodiment pertaining to fixation device 10, fixation device 20′ can vary from that shown, including, but not limited to, in its shape, the shape of its components and the direction in which its cart elements can slide.

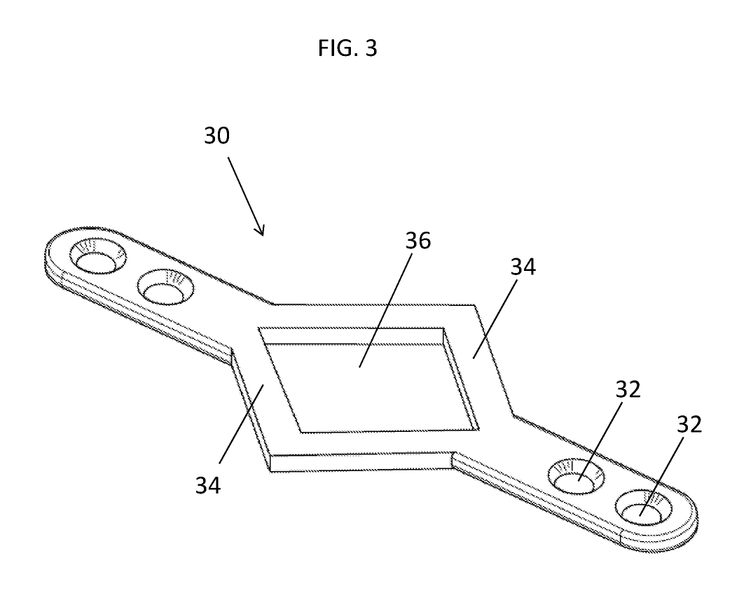

Referring to FIG. 3 there is shown yet a further embodiment of a bone fixation device 30. Bone fixation device 30 includes a bone contacting surface and an upper surface facing away from the bone with a plurality of holes or apertures 32 extending between the two surfaces. Holes or apertures 32 may be either threaded (for use with locking screws) or non-threaded (for use with non-locking or compression screws) and may be any suitable shape, such as circular or oblong. Bone fixation device 30 includes arm extensions 34 that connect opposing ends of the bone fixation device 30 and which form an opening 36. The shape of opening 36 is dependent on the shape of the arm extensions 34 and could be, by way of example and not limitation, diamond (as shown) or “O” shaped. Screws are inserted through the holes or apertures 32 into a bone such that both the fracture and the opening 36 are flanked on each side by at least one screw. The arm extensions 34 and opening 36 provide for resistance against torsion or bending while allowing for axial dynamization along the main axis of the bone fixation device 30. In other words, if screws are fixed into a bone through holes or apertures 32 on opposing sides of the opening 36, the arm extensions 34 can flex and allow for the bone to move along the main or longitudinal axis of the bone fixation device 30 while the screws remain rigidly fixed in the bone through holes or apertures 32. Preferably, the arm extensions 34 are constructed such that the two arm extensions 34 have a similar thickness as each other and as the rest of the body of the bone fixation device 30. This allows for the same bending rigidity in a plane through the bone fixation device 30. Again, it is to be understood that fixation device 30 may vary, including, but not limited to, in the shape of its components and its intended use.

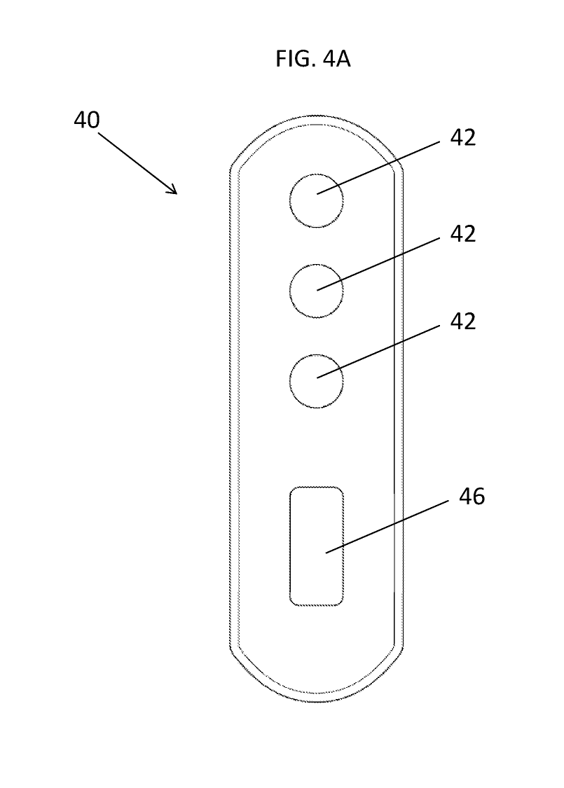

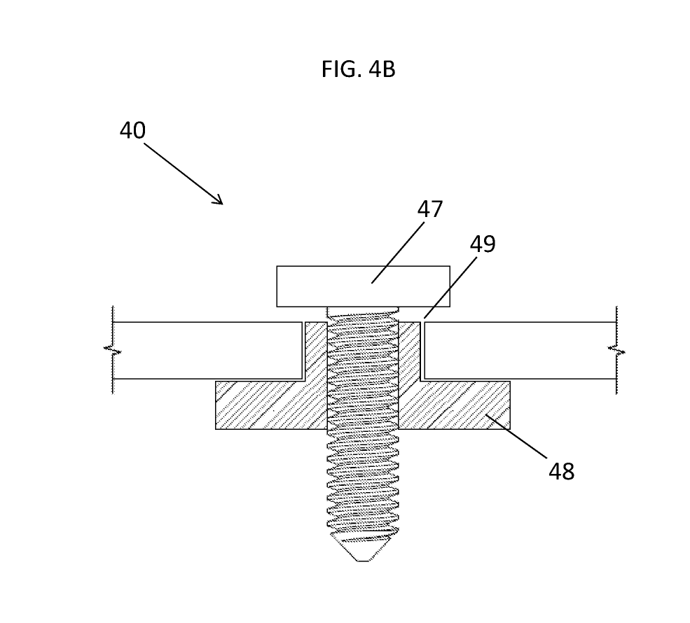

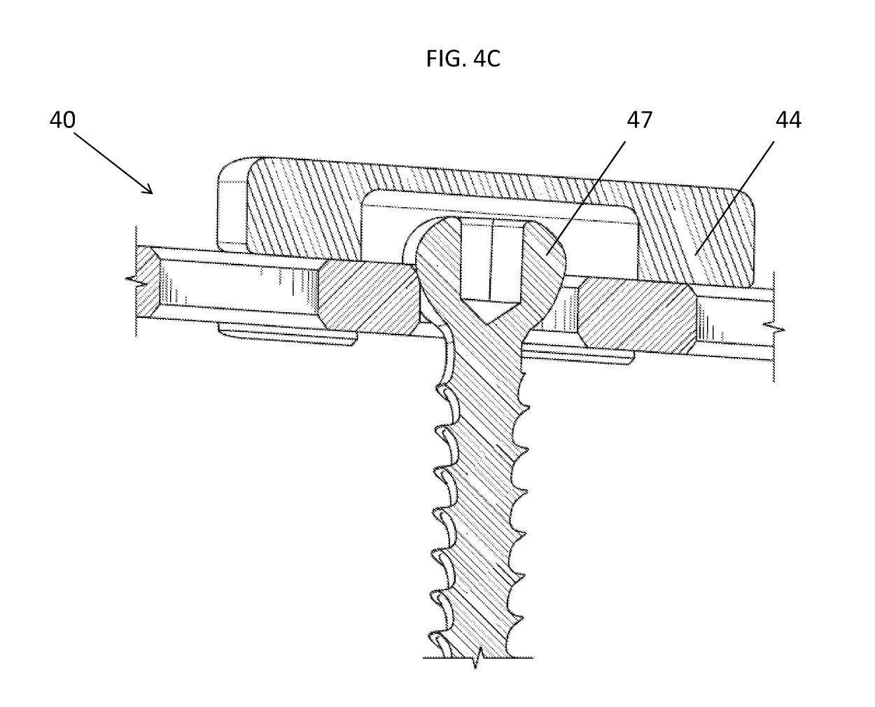

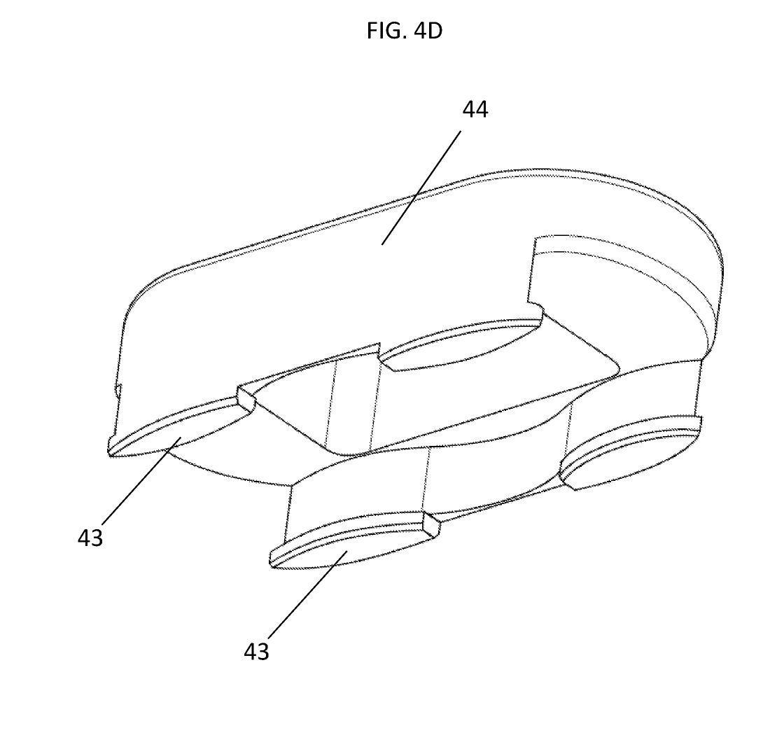

Referring to FIG. 4A, there is shown another embodiment of a bone fixation device 40. Bone fixation device 40 includes a bone contacting surface and an upper surface facing away from the bone with a plurality of holes or apertures 42 extending between the two surfaces. Holes or apertures 42 may be either threaded (for use with locking screws) or non-threaded (for use with non-locking or compression screws) and may be any suitable shape, such as circular or oblong. Bone fixation device 40 also includes at lease one oblong hole or aperture 46. During implantation of the bone fixation device 40 onto a bone across a fracture site, at least one hole or aperture 42 has a screw inserted into it and through the bone, and a screw is inserted into at least one oblong hole or aperture 46 on the opposite side of the fracture site. The oblong shape of the hole or aperture 46 allows the bone and screw to move in the direction of the main or longitudinal axis of the bone fixation device 40. Preferably, the screw includes a mechanism to limit the ability of the screw to tilt in the plate while still allowing the screw to slide within the oblong hole or aperture 46 in the direction of the main or longitudinal axis of the bone fixation device 40, as will be described more fully below.

For instance, referring to FIG. 4B, there is shown a mechanism to allow a screw 47 to slide within the oblong hole or aperture 46 in the direction of the main or longitudinal axis of the bone fixation device 40, while restricting tilting or movement of the screw 47 in other directions. In this illustrative embodiment, a screw 47 is inserted into the oblong hole or aperture 46 and through a nut 48 that at least partially sits under the bone fixation device 40. Similar to the apertures 46, the nut 48 may include an aperture that is either threaded or non-threaded. In the illustrated embodiment, the nut 48 has a top portion and a bottom portion. The bottom portion of the nut 48 is in the nature of flanges that have a width. The width of the flanges is greater than the major axis of the oblong aperture 46 such that the flanges extend beyond the aperture 46 of the bone fixation device 40 to limit the ability of screw 47 to tilt in relation to the bone fixation device. The top portion of nut 48 had a width. The width of the top portion of the nut is smaller than the major axis of the oblong aperture 46 such that the top portion fits within the oblong aperture, allowing for the screw 47 and nut 48 to move within the gap spaces 49 between the nut and the ends of oblong hole or aperture. This configuration allows for micromotion of the bone fragments in the direction of the main or longitudinal axis of the bone fixation device 40 to promote healing while restricting bending, torsion or movement in other directions which could hinder healing of the bone fracture. The bottom flanges of nut 48 may be various shapes or sizes and are largely a matter of design choice. For example, the bottom flanges of nut 48 are preferably thin relative to the thickness of the bone fixation device 40 to provide a more flush contact between the bone fixation device and the bone. In an alternate embodiment, the nut 48 can be a thin disc with a hole to accept a screw 47. In this embodiment, the gap spaces 49 exist between the screw 47 and the bone fixation device 40, rather than between the nut 48 and the bone fixation device.

Referring to FIG. 4C-4D, there is shown another embodiment of a mechanism to allow a screw 47 to slide within the oblong hole or aperture 46 in the direction of the main or longitudinal axis of the bone fixation device 40, while restricting tilting or movement of the screw 47 in other directions. In this illustrative embodiment, a screw 47 is inserted into the oblong hole or aperture 46 and a screw cap 44 is connected to the bone fixation device 40 over the screw 47. The screw cap 44 can have a generally oblong shaped recess in the underside of the top of the screw cap 47 to further facilitate sliding of the screw 47 along the main axis of the bone fixation device 40. The screw head and the top of the screw thread of screw 47 constrains motion of the screw 47 in the direction of the short axis of the bone fixation device 50 (i.e., in the direction of the minor axis of the oblong hole or aperture 46). The screw cap additionally prevents the screw 47 from tilting in other directions relative to the bone fixation device 40. The screw cap 44 may lock or otherwise engage the bone fixation device 40, for example by way of snapping bottom portions 43 of the screw cap 44 to the bone contacting surface of the bone fixation device 40, which acts to further constrain the motion of the screw 47 in a direction normal to the bone fixation device 40. The bottom portions 43 of the screw cap 44 may be flanges extending inwards from the side walls of the screw cap in the direction toward the aperture 46 of the bone fixation device 40. In the illustrated embodiment, the screw cap 44 includes two side walls projecting down from the top of the screw cap, and each side wall has two bottom portions 43 in the nature of flanges that engage the bone contacting surface of the bone fixation device 40. The flanges extend generally parallel to the plane of the bone fixation device 40 and the top surface of the screw cap 44. The outer surface of each side wall in the illustrated embodiment is generally orthogonal to the top surface of the screw cap 44. The inner surface of each side wall may be of varying thickness. In the illustrated embodiment, the center of each side wall is relatively thin, while the outer portions of each side wall are relatively thick. The inner surface of each side wall may be rounded or otherwise contoured to provide clearance for the head of a screw 47. In alternate embodiments, the two side walls could each have a single bottom portion 43 in the nature of a flange or other supporting structure to mate with the bone fixation device 40. For example, the bone contacting surface of the bone plate 40 could alternately include notches or grooves and the side walls of the screw cap 44 could include protrusions to mate with the notches or grooves. It is to be understood that different caps 44 can be provided to allow for different movement of the screws. For example, the size and shape of the recess in the screw cap 44 may be altered to change the ability of the screw 47 housed therein to move different magnitudes in different directions. Similarly, the screw cap 44 and recess formed therein could take the form of other shapes than oblong, such as rectangular or square shapes. The screw cap 44 can be formed of a metal, plastic, or other material suitable for implantation in the human body.

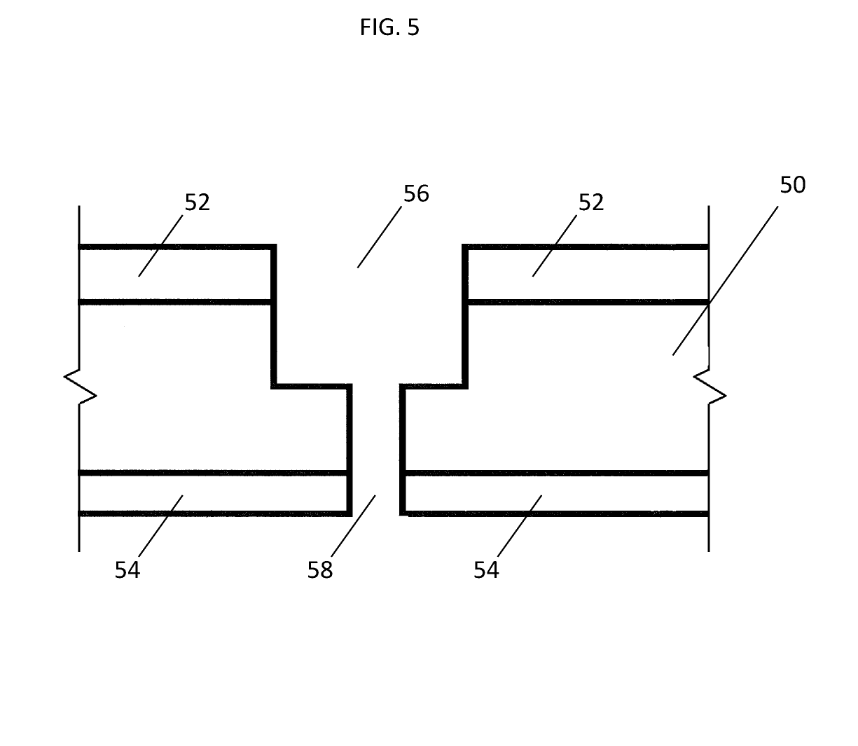

Referring now to FIG. 5, there is shown a cross section of a bone 50 along the main or longitudinal axis of the bone after a drilling procedure has been performed. In the method, a bone hole extension 56 is created in the near cortex 52 by overdrilling, for example by milling or sonic drilling. In addition, a relatively small drill hole 58 is created in the far cortex 54 of the bone 50. If a bone fixation device with an oblong aperture, such as the embodiment illustrated in FIG. 4A, is implanted onto the bone 50 and a screw is inserted through the oblong hole of the bone fixation device and through the hole extension 56 and further through the relatively small drill hole 58, the rigidity of the screw and bone 50 construct is reduced by allowing movement of the screw within the bone hole extension 56. The screw, either not being in contact or being in reduced surface contact with the bone 50 on the near cortex 52 side, could thus bend elastically in the bone hole extension 56. By creating the bone hole extension 56 in an oblong shape, a screw inserted into the bone hole extension 56 and further through the relatively small drill hole 58 could move in the direction of the main axis of the bone 50 while limiting the torsion, bending, or movement of the bone in other directions.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. (canceled)

2. A bone fixation device comprising:
a first bone contacting surface and a second surface opposite the first surface;
a first plate portion extending to a first end of the device and a second plate portion extending to a second end of the device;
a first aperture extending through the first and second surfaces on the first plate portion and a second aperture extending through the first and second surfaces on the second plate portion of the device;
a first flexible arm extending from the first plate portion toward the second plate portion; and
a second flexible arm extending from the second plate portion toward the first plate portion and being connected to the first flexible arm, the first and second arms allowing the first and second ends or the device to move toward and away from each other.

3. The bone fixation device of claim 2, wherein the first flexible arm includes a first pair of arm members and the second flexible arm includes a second pair of arm members.

4. The bone fixation device of claim 3, wherein the first pair of arm members and the second pair of arm members define an open space therebetween.

5. The bone fixation device of claim 4, wherein the open space is generally diamond-shaped.

6. The bone fixation device of claim 2, wherein the first plate portion, second plate portion, first flexible arm, and second flexible arm each have a substantially equal thickness.

7. The bone fixation device of claim 3, wherein the first pair of arm members includes a first arm extension extending at a first oblique angle relative to the first plate portion and a second arm extension extending at a second oblique angle relative to the first plate portion, and the second pair of arm members includes a third arm extension extending at a third oblique angle relative to the second plate portion and a fourth arm extension extending at a fourth oblique angle relative to the second plate portion.

8. The bone fixation device of claim 7, wherein the first, second, third, and fourth oblique angles are substantially equal.

9. The bone fixation device of claim 4, wherein the first plate portion has a longitudinal axis extending therethrough and the second plate portion has a second longitudinal axis extending therethrough, the first and second longitudinal axes being the same and passing through the open space.

10. The bone fixation device of claim 9, wherein the first and second pairs of arm members are adapted to allow the bone fixation device to flex in the direction of the longitudinal axis while maintaining rigidity in other directions.

11. The bone fixation device of claim 2, wherein at least one of the first and second apertures is oblong.

12. The bone fixation device of claim 2, wherein at least one of the first and second apertures is threaded.

13. The bone fixation device of claim 2, wherein at least one of the first and second apertures is non-threaded.

14. A method of fixing a bone plate to a bone with a fracture comprising the steps of:
providing the bone plate including:
a first bone contacting surface and a second surface opposite the first surface;
a first plate portion extending to a first end of the device, and a second plate portion extending to a second end of the device opposite;
a first aperture extending through the first and second surfaces on the first plate portion and a second aperture extending through the first and second surfaces on the second plate portion;
a first flexible arm extending from the first plate portion toward the second plate portion; and
a second flexible arm extending from the second plate portion toward the first plate portion and being connected to the first flexible arm, the first and second arms allowing the first and second ends or the device to move toward and away from each other;
inserting a first fastener through the first aperture and into the bone on a first side of the fracture; and
inserting a second fastener through the second aperture and into the bone on a second side of the fracture opposite the first side.

15. The method of claim 14, wherein the first flexible arm includes a first pair of arm members and the second flexible arm includes a second pair of arm members.

16. The method of claim 15, wherein the first pair of arm members and the second pair of arm members define an open space therebetween.

17. The method of claim 14, wherein the first fastener is a locking screw and the first aperture is threaded.

18. The method of claim 14 wherein the first aperture is non-threaded.
